SEQUENCE LISTING (1 sequence)

SEQ ID NO: 1 - DNA, 1278 bp, CHLORELLA VARIABILIS
atggctggaacggcgacagagagcctcaccaggtcgcggagcgcgctgcaccgactggcttgcagcgctcaaaactatgcctgggggcgccaccacgaggattcggaggtggcacagctggtggctgcatctgggcggcaagtggacgagtccaagccctatgccgagctgtggatgggcacccaccccgcggcgccctccctgctggcagacaacggctacgcggggcagccgctgctggcgctgctgcgcgaccggcccgagctgctgggcgccgcgctgccgcggtttggctgcgacctgcccttcctcttcaaggtgctctccgtgggcaccgccctctccatccagtcacaccccgacaaggcgctggcggagcggctgcacgcggcaaggccagaggtgtacaaggacgccaaccacaagccggagatggcgctggcgctgagcgagtttgaggcgctgtgcagctttgtgccgcacgaggagctggtggcggcgctgcgcgccgtgcccgagctggcggcctgctgcggcgaggcgcgcgtcgccgcgctggcggctgccgccccctgcggcgcgcagcggcggcaggcgctcaaggcggccttccacgccgtcatggcctgccccgccgagcgggcggcggagtgcgtgcgcgcgctgtgctcgcggctggagcgcgaggcggcagcggggcgccagctcagcgcgcgggagcagctgacgctgcgcctgcagcggcagtaccccggggacgtcggcgtgctggcctcctggttcctcaaccacctgcgcctgcgcccgggccaggcggtggcgctgcccgccaacgagccccacgcatacatctcgggagagattgtggagtgcatggccacgtcagacaacgtgatccgcgcggggctgacccccaagctgcgggatgtggagacgctgtgcgagtcgctgacgtaccggcagggggtgcccgaggtgatggagggcgccaagtcggcggcctcgccacacctggcctgctaccgcccgcccttcagggagtttgagatctggcgctacacgccgcccgccggctcccgcgaggcgctgccgccgcccgcgggcccgctgctcatgctggtgcagcagggcgcgatgcacgtgcgcagcggcgagcagtcgcgcctccttaagcggggcgacgtctactttgtggcggcgggcgcggagctgcagctggaggcgtcggccgatgtgtcggcctgggtgaccgcctgcaacggaatggcctttgagtga

(19) United States
(12) Patent Application Publication
(10) Pub. No.: US 2017/0303488 A1
(43) Pub. Date: Oct. 26, 2017

(54) CHLORELLA VARIABILIS-DERIVED PHOSPHOMANNOSE ISOMERASE GENE AND APPLICATION THEREOF

(71) Applicant: RICE RESEARCH INSTITUTE, ANHUI ACADEMY OF AGRICULTURAL SCIENCES, HEFEI, ANHUI (CN)

(72) Inventors: JIANBO YANG, HEFEI, ANHUI (CN); HAO LI, HEFEI, ANHUI (CN); PENGCHENG WEI, HEFEI, ANHUI (CN); LI LI, HEFEI, ANHUI (CN); YACHUN YANG, HEFEI, ANHUI (CN); DAHU NI, HEFEI, ANHUI (CN); JUAN LI, HEFEI, ANHUI (CN); RUIYING QIN, HEFEI, ANHUI (CN); HUI MA, HEFEI, ANHUI (CN)

(21) Appl. No.: 15/524,610

(22) PCT Filed: May 25, 2015
(86) PCT No.: PCT/CN2015/079651

§ 371 Date: May 4, 2017

(30) Foreign Application Priority Data
Jan. 5, 2015 (CN) 201510002583.6

(51) Int. Cl. A01H 5/02; C12N 15/74; C12N 15/70; C12N 9/90; C12N 5/14; A01H 5/12; A01H 5/10; A01H 5/06; C12N 15/82; C12N 9/00; A01H 5/00

(52) U.S. Cl. CPC A01H 5/02; A01H 5/06; C12N 15/74; C12N 15/70; C12N 9/90; C12N 15/82; C12N 5/14; A01H 5/12; A01H 5/10; A01H 5/00; C12N 9/00

(57) ABSTRACT

The present invention provides a Chlorella variabilis-derived phosphomannose isomerase gene, herein named ChloPMI. The present invention also provides a prokaryotic expression vector comprising ChloPMI, which can be used for identifying mannose metabolic activity of ChloPMI protein. Further, the present invention provides an expression cassette and a plant expression vector comprising ChloPMI, and a use of the expression cassette and the expression vector in genetic transformation of plants. According to the present invention, the transformation of rice cells is successfully achieved with the plant expression vector constructed from the ChloPMI gene using mannose as a selection agent. According to the present invention, a plant-derived phosphomannose isomerase gene is successfully separated and cloned from Chlorella variabilis. Since the plant-derived phosphomannose isomerase gene is derived from Chlorella variabilis, it is environment-friendly and has no potential hazard to human, which is very beneficial in promoting and applying transgenic products and eliminating any existing doubts on transgenes.

### BACKGROUND OF THE INVENTION

### 1. Technical Field

The present invention relates to the field of biotechnology and plant genetic engineering technology. In particular, the present invention relates to separation, cloning and use of a Chlorella variabilis-derived phosphomannose isomerase gene ChloPMI.

### 2. Description of Related Art

Transgenic technology is an effective directional plant improvement method developed in early 1980s. The technology achieves rapid directional improvement of a target trait by introducing a target gene into a recipient with Agrobacterium-mediated transformation, gene gun transformation and other methods, and performing marker screening and molecular detection to obtain a stably expressed transformant. Transgenic technology provides a new path and opens up a new horizon for output promotion, quality improvement and increased resistance of crops.

An antibiotic marker gene is a main safety issue caused by genetically modified foods to human health. The existing transgenic technology primarily uses the antibiotic resistance gene as a selection marker. The antibiotic marker gene is transformed into a target crop with a target gene to be inserted, so as to assist in screening and identifying transformed cells, tissues and regenerated plants in genetic transformation of plants. The marker gene itself has no safety issue. However, such a gene may enter intestinal tract with foods, where a potential risk exists that drug-resistant strains are produced due to genetic exchange with intestinal microbes, to affect the medical effect of antibiotics. In order to replace antibiotic resistance genes, other types of selection marker gene, such as herbicide resistance genes, amino acid metabolism selection genes, visual marker genes and the like, have been successively put under research and development. However, these selective marker genes either suffer from the similar problem or have a screening efficiency and cost not suitable for large-scale applications.

In contrast, phosphomannose isomerase is a glucose metabolism gene. Although higher plant cells such as rice cells can convert mannose into mannose 6-phosphate, the cells cannot further convert mannose 6-phosphate into fructose 6-phosphate for utilization in the glycolysis pathway, due to the lack of phosphomannose isomerase in themselves. Therefore, phosphomannose isomerase may serve as a selective marker gene. In contract to negative selection for antibiotics, herbicides and the like, positive selection is performed for mannose. Transformed cells express phosphomannose isomerase, and may grow normally by using mannose as a carbon source. The screening efficiency is thus high. In addition, fructose 6-phosphate, as a catalysate of phosphomannose isomerase, is a main ingredient of honey and pulp, both of which are environmental-friendly nature substances.

However, currently widely used phosphomannose isomerase is isolated from prokaryotic E. coli, which may adversely affect transformation of a recipient genome, and may also lead to concerns about its safety.

### SUMMARY OF THE INVENTION

In view of the above problems, the present invention is intended to separate a plant-derived phosphomannose isomerase gene. In particular, through continuous attempts, the present inventors finally separate and clone a phosphomannose isomerase gene ChloPMI from Chlorella variabilis. Further, the present invention also constructs a prokaryotic expression vector comprising ChloPMI for use in identifying mannose metabolic activity of ChloPMI protein; and constructs a plant expression vector comprising ChloPMI for use in genetic transformation of plants with ChloPMI as a selection marker.

Specifically, in a first aspect, the present invention provides a Chlorella variabilis-derived phosphomannose isomerase gene having a nucleotide sequence represented by SEQ ID NO: 1. For convenience of expression, it is named ChloPMI in the present invention.

In a second aspect, the present invention provides a method for identifying mannose metabolic activity of ChloPMI protein, comprising the steps of: designing a ChloPMI prokaryotic expression primer; obtaining a prokaryotic expression vector fused to GST (glutathione S-transferase) fragment via subcloning; transforming the prokaryotic expression vector into E. coli expression strain BL21; and identifying the activity of ChloPMI by a color reaction with phenol red.

In a third aspect, the present invention provides a plant expression vector comprising the ChloPMI gene. In the construction process, with Xho I restriction site, a pCAMBIA1381 vector is digested with Xho I and recovered. Since Xho I restriction site is added at both ends of the synthesized ChloPMI sequence, ChloPMI may be ligated to the pCAMBIA1381 vector using T₄ ligase, obtaining a plant expression vector pCAMBIA1381-ChloPMI.

In another aspect, the present invention provides an expression cassette characterized in that: the expression cassette comprises the above-described ChloPMI gene.

In another aspect, the present invention provides a method for obtaining transformed rice cells with mannose as a carbon source using the pCAMBIA1381-ChloPMI expression vector, comprising the steps of:

(1) separating embryos from sterilized rice seeds with shell removed and placing the embryos on callus induction medium to generate secondary callus;

(2) transferring the secondary callus to a new callus induction medium for pre-culture;

(3) contacting the callus obtained in the step (2) with Agrobacterium carrying the ChloPMI selective marker gene for 15 min;

(4) transferring the callus from the step (3) into a culture dish (2.5-3.5 mL Agrobacterium suspension medium is added) lined with three sterile filter papers, and culturing for 48 h at 21-23° C.;

(5) placing the callus from the step (4) on a pre-selection medium, and culturing for 5-7 days; and

(6) transferring the callus from the step (5) onto a selection medium containing mannose to obtain resistant rice callus (rice cells) using mannose as a carbon source.

The seeds in the step (1) are mature seeds; the induction medium in the steps (1) and (2) is the induction medium listed in Table 1 herein; the contacting with Agrobacterium in the step (3) refers to immersing the callus in the Agrobacterium suspension; the Agrobacterium suspension medium in the step (4) is the suspension medium listed in Table 1 herein; the pre-selection medium in the step (5) is the pre-selection medium listed in Table 1 herein; and the selection medium in the step (6) is the selection medium listed in Table 1 herein.

In a preferred embodiment, the rice is japonica rice, more preferably, japonica rice cultivar Nipponbare.

In another aspect, the present invention provides a use of the above-described gene, expression cassette or vector, characterized in that: transformed plant cells are obtained by the above-described method using the ChloPMI gene as a selection marker, and the transformed plant cells are used to obtain a transgenic plant or plant part.

Preferably, the plant includes a cereal crop, vegetable crop, flower crop, and energy crop.

Preferably, the plant part includes a cell, protoplast, cell and tissue culture, callus, cell mass, plumule, pollen, ovule, petal, style, stamen, leaf, root, root tip, anther, and seed.

Exemplary formulations of media for use in the present invention in a preferred embodiment are given in Table 1 below. A person skilled in the art will understand that, each medium in the present invention may be an ordinary medium, in addition to that having a particular formulation shown below, and the ordinary medium can also achieve the objects of the present invention, but there is some difference in effect.

TABLE 1
Exemplary formulations of media

| Medium use | Formulation |
|---|---|
| Callus induction | Optimized N6 macro-elements ([NO₃⁻]/[NH₄⁺] = 40 mM/10 mM), |
| and pre-culture | B5 micro-elements, MS iron salts, B5 vitamins, 500 mg/L |
| | proline, 500 mg/L glutamine, 300 mg/L casein |
| | hydrolysate, 30 g/L sucrose, 2 mg/L 2,4-D (2,4- |
| | dichlorophenoxyacetic acid), 3 g/L phytagel, pH 5.80 |
| Agrobacterium | 5 g/L yeast extract, 10 g/L peptone, 10 g/L NaCl, 15 g/L |
| culture | Bacto-agar, 50 mg/L kanamycin, pH 7.20 |
| Agrobacterium | Optimized N6 macro-elements ([NO₃⁻]/[NH₄⁺] = 40 mM/10 mM), |
| suspension and | B5 micro-elements, MS iron salts, B5 vitamins, 500 mg/L |
| co-culture | proline, 500 mg/L casein hydrolysate, 2 mg/L 2,4-D, 20 g/L |
| | sucrose, 10 g/L glucose, 100 μmol/L acetosyringone, pH |
| | 5.20 |
| Pre-screening | Basic components are similar to those of the callus induction |
| | medium, except for the addition of 250 mg/L carbenicillin |
| Screening | Optimized N6 macro-elements ([NO₃⁻]/[NH₄⁺] = 40 mM/10 mM), |
| | B5 micro-elements, MS iron salts, B5 vitamins, 500 mg/L |
| | proline, 500 mg/L glutamine, 300 mg/L casein |
| | hydrolysate, 5 g/L sucrose, 12.5 g/L mannose, 2 mg/L 2,4-D, |
| | 250 mg/L carbenicillin, 3 g/L phytagel, pH 5.80 |

“Optimized N6 macro-elements” referred to in the table means that [NO₃⁻]/[NH₄^{+]}=40 mM/10 mM in the N6 macro-elements.

In a preferred embodiment, the nucleotide sequence of the ChloPMI gene is a nucleotide sequence represented by SEQ ID NO: 1, particularly:

| | |
|---|---|
| atggctggaa cggcgacaga gagcctcacc aggtcgcgga gcgcgctgca ccgactggct | 60 |
| tgcagcgctc aaaactatgc ctgggggcgc caccacgagg attcggaggt ggcacagctg | 120 |
| gtggctgcat ctgggcggca agtggacgag tccaagccct atgccgagct gtggatgggc | 180 |
| acccaccccg cggcgccctc cctgctggca gacaacggct acgcggggca gccgctgctg | 240 |
| gcgctgctgc gcgaccggcc cgagctgctg ggcgccgcgc tgccgcggtt tggctgcgac | 300 |
| ctgcccttcc tcttcaaggt gctctccgtg ggcaccgccc tctccatcca gtcacacccc | 360 |
| gacaaggcgc tggcggagcg gctgcacgcg gcaaggccag aggtgtacaa ggacgccaac | 420 |
| cacaagccgg agatggcgct ggcgctgagc gagtttgagg cgctgtgcag ctttgtgccg | 480 |
| cacgaggagc tggtggcggc gctgcgcgcc gtgcccgagc tggcggcctg ctgcggcgag | 540 |
| gcgcgcgtcg ccgcgctggc ggctgccgcc ccctgcggcg cgcagcggcg gcaggcgctc | 600 |
| aaggcggcct tccacgccgt catggcctgc cccgccgagc gggcggcgga gtgcgtgcgc | 660 |
| gcgctgtgct cgcggctgga gcgcgaggcg gcagcggggc gccagctcag cgcgcgggag | 720 |
| cagctgacgc tgcgcctgca gcggcagtac cccggggacg tcggcgtgct ggcctcctgg | 780 |
| ttcctcaacc acctgcgcct gcgcccgggc caggcggtgg cgctgcccgc caacgagccc | 840 |
| cacgcataca tctcgggaga gattgtggag tgcatggcca cgtcagacaa cgtgatccgc | 900 |
| gcggggctga cccccaagct gcgggatgtg gagacgctgt gcgagtcgct gacgtaccgg | 960 |
| cagggggtgc ccgaggtgat ggagggcgcc aagtcggcgg cctcgccaca cctggcctgc | 1020 |
| taccgcccgc ccttcaggga gtttgagatc tggcgctaca cgccgcccgc cggctcccgc | 1080 |
| gaggcgctgc cgccgcccgc gggcccgctg ctcatgctgg tgcagcaggg cgcgatgcac | 1140 |
| gtgcgcagcg gcgagcagtc gcgcctcctt aagcggggcg acgtctactt tgtggcggcg | 1200 |
| ggcgcggagc tgcagctgga ggcgtcggcc gatgtgtcgg cctgggtgac cgcctgcaac | 1260 |
| ggaatggcct ttgagtga | 1278 |

According to the present invention, a phosphomannose isomerase gene is successfully separated from Chlorella variabilis, such that a plant-derived phosphomannose isomerase gene is obtained for the first time. Since Chlorella variabilis is an environment-friendly natural substance and has no potential hazard to human, this is very beneficial in promoting and applying transgenic products, eliminating any existing doubts in safety on genetically modified foods, and addressing potential threats probably resulting from the antibiotic marker genes.

Further, the present invention also provides a prokaryotic expression vector comprising ChloPMI for use in identifying mannose matabolic activity of ChloPMI protein; and constructs a plant expression vector comprising ChloPMI for use in genetic transformation of plants using ChloPMI as a selection marker.

### BRIEF DESCRIPTION OF THE DRAWINGS

FIG. 1 shows a schematic illustration of a pCAMBIA1381-ChloPMI vector plasmid constructed using the phosphomannose isomerase gene of the present invention.

FIG. 2 shows a picture of identifying ChloPMI activity through a staining reaction, where NC (negative control) is E. coli expressing strain BL21 transformed with a pGEX-6P-1 empty vector; 1 is BL21 strain containing Chlorella variabilis ChloPMI expression vector pGEX-ChloPMI; and 2 is BL21 strain containing E. coli PMI expression vector pGEX-PMI.

FIG. 3 shows a picture of resistant rice callus produced by a mannose-based selection after transformation with Agrobacterium containing a pCAMBIA1381-ChloPMI plasmid.

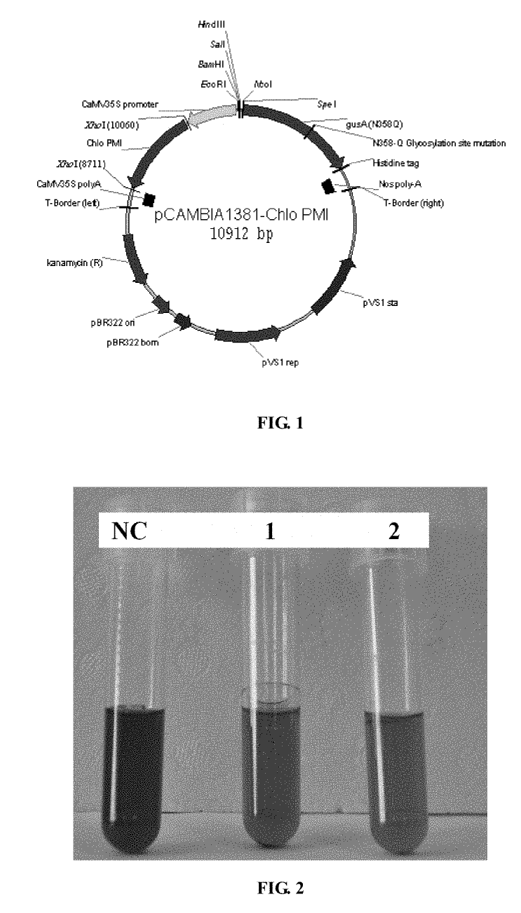

### DETAILED DESCRIPTION OF THE PREFERRED EMBODIMENTS

Unless otherwise specified, the operations in specific embodiments described below are performed following general conventional operations in the art. A person skilled in the art can easily obtain the teaching regarding such conventional operations from the prior art, for example, can make reference to the textbook, Sambrook and David Russell, Molecular Cloning: A Laboratory Manual, 3rd ed., Vols 1, 2; Charles Neal Stewart, Alisher Touraev, Vitaly Citovsky and Tzvi Tzfira, Plant Transformation Technologies and the like. Unless specifically stated, medicinal raw materials and reagent materials used in the examples below are commercially available products.

Specific embodiments of the present invention will be described below in detail with reference to the accompanying drawings. It should be noted that the figures of experimental results illustrated in the drawings are originally colored diagrams; however, in view of the provisions in the patent law, the applicant converts them to grayscale images, and despite this, the difference between the experimental results under different conditions still can be distinguished from shades in the images.

### EXAMPLE 1

### Obtaining and Cloning a ChloPMI Gene

A sequence with the highest homology, namely, the ChloPMI protein sequence, was obtained by aligning homologous sequences in the genomic sequence (genome.jgi-psf.org) of Chlorella variabilis which can utilize mannose based on phosphomannose isomerase protein sequence of the bacteria. The phosphomannose isomerase protein sequence of the bacteria is as follows:

MQKLINSVQNYAWGSKTALTELYGMENPSSQPMAELWMGAHPKSSSR
VQNAAGDIVSLRDVIESDKSTLLGEAVAKRFGELPFLFKVLCAAQPL
SIQVHPNKHNSEIGFAKENAAGIPMDAAERNYKDPNHKPELVFALTP
FLAMNAFREFSEIVSLLQPVAGAHPAIAHFLQQPDAERLSELFASLL
NMQGEEKSRALAILKSALDSQQGEPWQTIRLISEFYPEDSGLFSPLL
LNVVKLNPGEAMFLFAETPHAYLQGVALEVMANSDNLRAGLTPKYID
IPELVANVKFEAKPANQLLTQPVKQGAELDFPIPVDDFAFSLHDLSD
KETTISQQSAAILFCVEGDATLWKGSQQLQLKPGESAFIAANESPVT
VKGHGRLARVYNKL

Thereafter, RNA of Chlorella variabilis was further extracted and reverse transcribed into cDNA. According to the coding sequence (CDS) of ChloPMI, gene specific cloning primers, a forward primer 5′-ATGGCTGGAACGGCGACAGAGA-3′ and a reverse primer 5′-TCACTCAAAGGCCATTCCGTTG-3′ were designed, and then PCR amplification was performed using the cDNA as a template.

PCR-amplified target fragments having a fragment length of 1278 bp were recovered, and were ligated to PGEM-T-Easy vector (available from Promega Inc.) according to the instructions for the vector. After the transformation of E. coli competent cells XL-Blue using thermal stimulation, positive clones were obtained via colony PCR screening. The identified positive clones were delivered to Invitrogen Inc. for sequencing. The correct clones by verification were recombinant plasmids containing ChloPMI, named as PGEM-T-ChloPMI. The nucleotide sequence of ChloPMI was represented by SEQ ID NO: 1.

### EXAMPLE 2

### Construction of a Prokaryotic Expression Vector Containing the ChloPMI Gene

PCR amplification was performed by the design of ChloPMI prokaryotic expression primers, a forward primer 5′-GGATCC ATGGCTGGAACGGCGACAGAGA -3′ (underline indicates BamHI restriction site) and a reverse primer 5′-CTCGAGCTCAAAGGCCATTCCGTTG-3′ (underline indicates XhoI restriction site) using the PGEM-T-ChloPMI recombinant plasmid as a template. The prokaryotic expression vector pGEX-ChloPMI fused to GST (glutathione-S-transferase) fragment was then obtained by ligating the recovered PCR-amplified target fragments to pGEX-6P-1 expression vector (available from GE Inc.) digested with BamHI and XhoI, and was transformed into E. coli expressing strain BL21. Simultaneously, a pGEX-6P-1 empty vector and pGEX-PMI containing E. coli phosphomannose isomerase expression vector were also transformed into E. coli expressing strain BL21, respectively.

### EXAMPLE 3

### ChloPMI Activity Analysis

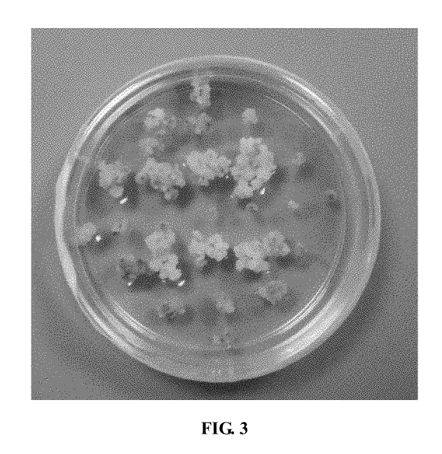

The BL21 strain containing the prokaryotic expression vector pGEX-ChloPMI, pGEX-PMI and pGEX-6P-1 empty vector was streaked. Monoclones were picked and inoculated into LB liquid medium (see Table 1 for composition, Agrobacterium culture medium, no agar), and cultured overnight with shaking at 37° C. (200 r/min). On the next day, the culture was centrifuged for 1 min at 6000 r/min at room temperature, the supernatant was discarded and the pellet was resuspended with a small amount of sterilized water. The resuspension was taken and plated on sterilized phenol red chromogenic medium (1% peptone, 0.5% NaCl, 50 mg/L phenol red, 30% mannose, PH 7.4) with 1:50. After 48 h, the change in medium color was observed. If the strain has an ability to metabolize mannose, the medium was acidified, resulting in a drop in PH value. The medium color therefore gradually changed into yellow from red at PH 7.4. Results from activity analysis of different vectors were seen in FIG. 2. As can be seen from the figure, NC strain (E. coli expressing strain BL21 transformed with the pGEX-6P-1 empty vector) does not have the ability to metabolize mannose, as the medium color is still red. In contract, E. coli of 1 (BL21 strain containing Chlorella variabilis ChloPMI expression vector pGEX-ChloPMI) and 2 (BL21 strain containing E. coli PMI expression vector pGEX-PMI) have an ability to metabolize mannose, such that the medium is acidified, resulting in a drop in PH value, and the color in medium thus gradually changes into yellow from red at PH 7.4.

### EXAMPLE 4

### Construction of a ChloPMI Plant Expression Vector

PCR amplification was performed by a forward primer 5′-CTCGAGATGGCTGGAACGGCGACAGAGA -3′ (underline indicates XhoI restriction site) and a reverse primer 5′-CTCGAGTCACTCAAAGGCCATTCCGTTG-3′ (underline indicates XhoI restriction site) using the PGEM-T-ChloPMI recombinant plasmid as a template. The plant expression vector pCAMBIA1381-ChloPMl was then obtained by ligating the recovered PCR-amplified target fragments to pCAMBIA1381 vector linearized with XhoI via T₄ ligase (FIG. 2). The plant expression vector was transformed into Agrobacterium tumefaciens strain EHA105 (deposited with Rice Research Institute, Anhui Academy of Agricultural Sciences) using the freeze-thaw method for genetic transformation.

### Example 5

### Genetic Transformation in Rice using ChloPMI as a Selective Marker Gene

1. Induction and Pre-Culture of Mature Embryo Callus

Mature seeds of Nipponbare (deposited with Rice Research Institute, Anhui Academy of Agricultural Sciences) were peeled. The normal appearance, clean and free of mildew seeds were selected and shaken for 90 sec in the presence of 70% alcohol, and then, the alcohol was poured off. The seeds were further washed with 50% sodium hypochlorite solution containing Tween20 (available chlorine concentration of stock solution >4%, 1 drop Tween20 per 100 ml), and shaken for 45 min (180 r/min) on a shaking table. Sodium hypochlorite was poured off. The seeds were washed 5-10 times with sterilized water until no odour of sodium hypochlorite. Finally, the seeds were added with sterilized water, and soaked overnight at 30° C. Embryos were separated along aleurone layer with a surgical blade, placed with scutellum up on induction medium (see Table 1 for composition) at 12 embryos/dish, and cultured in the dark at 30° C. to induce the callus.

Two weeks later, spherical, rough, pale yellow secondary callus appeared. Pre-culture operation could be performed, that is, the secondary callus were transferred onto a new callus induction medium, and pre-cultured in the dark at 30° C. for 5 d. After the completion of the pre-culture, small particles having a good condition and a strong split were collected with a spoon into a 50 mL sterile centrifuge tube for Agrobacterium infection.

2. Culture of Agrobacterium Strains and Preparation of Suspensions

Agrobacterium strain EHA105 (deposited with Rice Research Institute, Anhui Academy of Agricultural Sciences) containing pCAMBIA1381-ChloPMI vector was streaked on an LB plate containing 50 mg/L kanamycin (see Table 1 for composition), and cultured in the dark at 28° C. 24 h later, the activated Agrobacterium was inoculated with a sterile inoculation loop onto a fresh LB plate containing 50 mg/L kanamycin for a second activation, and cultured overnight in the dark at 28° C. 20-30 mL of Agrobacterium suspension medium (see Table 1 for composition) was added in a 50 mL sterile centrifuge tube. The Agrobacterium activated twice was scraped off by the inoculation loop, with optical density 660 nm (OD660) adjusted to about 0.10-0.25, and allowed to stand for over 30 min at room temperature.

3. Infection and Co-Culture

The prepared callus (see step 1) were added with the Agrobacterium suspension, and soaked for 15 min with gently shaking from time to time. After the completion of soaking, the liquid was poured off (no liquid drops if possible). The excess Agrobacterium suspension on the callus surface was wicked away with sterile filter papers, and blow dried by sterile wind on an ultra-clean station. Three sterile filter papers were placed on a 100×25 mm disposable sterile culture dish, and 2.5 mL of the Agrobacterium suspension medium was added. The blotted callus were uniformly dispersed onto the filter papers, and cultured for 48 h in the dark at 23° C.

4. Pre-Screening and Screening Culture

After the completion of the co-culture, the co-cultured callus were uniformly dispersed in a pre-selection medium (see Table 1 for composition), and cultured for 5 d in the dark at 30° C. After the completion of the pre-screening culture, the callus were transferred onto a selection medium (see Table 1 for composition) at 25 callus/culture dish, and cultured in the dark at 30° C. 2-3 weeks later, the growth of resistant calli is evident (as shown in FIG. 3), and differentiated regeneration could be performed. As can be seen in FIG. 3, the newborn resistant callus assume a pale yellow color, compact texture and strong granular sensation, indicating that embryonic callus have a better state, and are suitable for subsequent differentiation and regeneration. It should be noted that the figures of experimental results illustrated in the drawings are originally colored diagrams; however, in view of the provisions in the patent law, the applicant converts them to grayscale images, and despite this, the difference between the experimental results under different conditions still can be distinguished from shades in the images.

Using the resulting resistant callus, a rice plant or plant part can be cultured.

It will be understood that the specific embodiments described herein are merely used for illustrative purposes to help a person skilled in the art to better understand the invention, and are not intended to limit the scope of the invention. Without departing from the spirit and scope of the invention, various changes or variations which may be made by a person skilled in the art according to the invention are intended to be within the scope of appended claims.

1. A phosphomannose isomerase gene from Chlorella variabilis having a nucleotide sequence represented by SEQ ID NO: 1.

2. A prokaryotic expression vector comprising the phosphomannose isomerase gene according to claim 1.

3. A prokaryotic identification method for identifying mannose metabolic activity of a phosphomannose isomerase gene, comprising:
performing color identification on an expression strain comprising the phosphomannose isomerase gene according to claim 1 by a color identification method with phenol red.

4. An expression cassette comprising the phosphomannose isomerase gene according to claim 1.

5. A plant expression vector comprising the phosphomannose isomerase gene according to claim 1.

6. A method for obtaining transformed rice cells by a mannose-based selection with a plant expression vector pCAMBIA1381-ChloPMI comprising the phosphomannose isomerase gene according to claim 1, comprising the steps of:
(1) separating embryos from sterilized rice seeds with shell removed and placing the embryos on callus induction medium to generate secondary callus;
(2) transferring the secondary callus to new callus induction medium for pre-culture to obtain the callus for the transformation;
(3) contacting the callus obtained in the step (2) with Agrobacterium for 15 min, wherein the Agrobacterium is incorporated with the plant expression vector carrying the phosphomannose isomerase gene;
(4) transferring the callus treated in the step (3) into a culture dish lined with a sterile filter paper, and culturing for 48 h at 21-23° C.;
(5) placing the callus treated in the step (4) on a pre-selection medium, and culturing for 5-7 days; and
(6) transferring the callus treated in the step (5) onto a selection medium to obtain resistant callus, that is, transformed rice cells which can metabolize mannose.

7. A use of the phosphomannose isomerase gene, the expression cassette and the plant expression vector, the phosphomannose isomerase gene from Chlorella variabilis having a nucleotide sequence represented by SEQ ID NO: 1, the expression cassette comprising the phosphomannose isomerase gene, the plant expression vector comprising the phosphomannose isomerase gene or the expression cassette, wherein transformed plant cells are obtained by the method according to claim 6 using the phosphomannose isomerase gene as a selection marker, and the resulting transformed plant cells are used to obtain a transgenic plant or plant part.

8. The use according to claim 7, wherein the plant comprises a cereal crop, vegetable crop, flower crop, and energy crop.

9. The use according to claim 7, wherein the plant part comprises a cell, protoplast, cell and tissue culture, callus, cell mass, plumule, pollen, ovule, petal, style, stamen, leaf, root, root tip, anther, and seed.

10. A prokaryotic identification method for identifying mannose metabolic activity of a phosphomannose isomerase gene, comprising: performing color identification on an expression strain comprising the prokaryotic expression vector according to claim 2 by a color identification method with phenol red.

11. A plant expression vector comprising the expression cassette according to claim 4.
